(19) United States
(12) Patent Application Publication
(10) Pub. No.: US 2007/0071809 A1
(43) Pub. Date: Mar. 29, 2007

(54) Ginger containing compositions to control pain

(76) Inventor: Vijay B. Vad, New York, NY (US)

Correspondence Address: WHYTE HIRSCHBOECK DUDEK S C, 555 EAST WELLS STREET, SUITE 1900, MILWAUKEE, WI, 53202, US

(21) Appl. No.: 11/235,675

(22) Filed: Sep. 26, 2005

(51) Int. Cl. A61K 36/906; A61K 31/737; A61K 31/7008; A61K 9/64

(52) U.S. Cl. 424/456; 514/54; 514/62; 424/756

(57) ABSTRACT

Pain is controlled by ingesting a composition comprising at least about 510 mg of ginger, at least about 1000 mg of glucosamine and at least about 1200 mg of chondroitin sulfate or methylsulfonylmethane, all weights based on a total daily dosage of the composition.

### BACKGROUND OF THE INVENTION

This invention relates generally to pain control, and more specifically to methods and compositions using ginger along with glucosamine and chondroitin sulfate or methylsulfonylmethane to enhance pain control and shorten onset of pain control effect in arthritis.

Glucosamine at 1500 mg per day has been shown to reduce knee pain in patients with osteoarthritis of the knee (Reginster JY et al: Long term effects of glucosamine sulfate on osteoarthritis progression and pain: A randomized place-controlled trial. Lancet 357: 251-256, 2001). The Reginster study examined 212 patients with half taking glucosamine and half taking placebo. The glucosamine group had statistically improved pain scores while the placebo group had worsened pain scores. Ginger at 510 mg per day has been shown to reduce knee pain in patients with osteoarthritis of the knee (Altman R et al: Effects of ginger on knee pain in patients with osteoarthritis. Arthritis and Rheumatism 44(11) 2531-2538, 2001(2)).

### BRIEF SUMMARY OF THE INVENTION

The invention is to increase the efficacy while shortening the pain control clinical effect of glucosamine by combining it with high levels of ginger. The technology is advanced due to combining these two therapeutic nutritional supplements for better and quicker pain control in treating arthritis symptoms.

In one embodiment the invention is a pain-controlling composition comprising at least about 510 mg of ginger, at least about 1000 mg of glucosamine and at least about 1200 mg of chondroitin sulfate or methylsulfonylmethane, all weights based on a total daily dosage of the composition.

In another embodiment the invention is a therapeutics delivery system comprising at least one capsule containing a composition comprising at least about 510 mg of ginger, at least about 1000 mg of glucosamine and at least about 1200 mg of chondroitin sulfate or methylsulfonylmethane, all weights based on a total daily dosage of the composition

Still another embodiment of the invention is a method for controlling pain, the method comprising ingesting a daily dosage of a composition that comprises at least about 510 mg of ginger, at least about 1000 mg of glucosamine and at least about 1200 mg of chondroitin sulfate or methylsulfonylmethane, all weights based on a total daily dosage of the composition.

### DETAILED DESCRIPTION OF THE INVENTION

The invention can be used as a nutritional supplement combining the therapeutic benefits of glucosamine along with ginger. The product is desirable due to increased and quicker pain control.

Glucosamine is thought to limit breakdown and breakdown products of cartilage leading to pain relief in arthritis. Ginger has been shown to have anti-inflammatory properties with reduction in pain by acting as a cyclooxygenase inhibitor. Thus glucosamine has a slower and different mechanism of action for pain control than does ginger. The addition of ginger markedly shortens the onset of pain reduction and since they both have different mechanisms of action, ginger with glucosamine also enhances the overall pain reduction effect. This has been observed in our clinical trial on over six hundred arthritic hips and knees which were finished in March of 2005. This invention leads us to conclude that ginger interacts with glucosamine very positively by decreasing duration of clinical effect of pain control while increasing the overall level of pain control. This invention differs from the prior art because it uses high doses of ginger from at least 510 mg up to 200 mg daily in combination with glucosamine.

Glucosamine and chondroitin sulfate are substances found naturally in the body. Glucosamine is a form of amino sugar that is believed to play a role in cartilage formation and repair. Chondroitin sulfate is part of a large protein molecule (proteoglycan) that gives cartilage elasticity.

Glucosamine has been used extensively and studied and published extensively for pain control in arthritis. Glucosamine is thought to limit cartilage breakdown and cartilage breakdown products which can cause pain and inflammation. Glucosamine is combined commonly with chondroitin sulfate or methylsulfonylmethane (MSM) which enhances its ability to limit cartilage breakdown. However, there may still not be enough pain relief for people wanting to be active.

Chondroitin sulfates provide the structural components of joint cartilage, inhibit free radical enzymes that degrade joint cartilage and collagen, and facilitate entry of glucosamine into inflamed joints. Like glucosamine, chondroitin sulfate attracts water into the cartilage matrix and stimulates the production of cartilage in addition to preventing enzymes from dissolving cartilage. Long-term treatment with chondroitin sulfate has been shown to reduce pain and increase range of motion. In addition, chondroitin sulfate has recently been shown to have potent antioxidant activity in addition to possessing anti-inflammatory properties.

The chondroitin sulfate family includes seven sub-types designated unsulfated chondroitin sulfate, oversulfated chondroitin sulfate and chondroitin sulfates A-E which vary in the number and position of their sulfate functional groups. Additionally, chondroitin sulfate B, also referred to as dermatan sulfate, differs in that iduronic acid is the predominant residue in the alternative hexuronic acid position. Chondroitin sulfates, A, B and C are the predominant forms found in mammals and may be involved in the modulation of various biological activities including cell differentiation, adhesion, enzymatic pathways and hormone interactions. The chondroitin sulfate in compositions of the present invention is preferably in powder form.

Both glucosamine and chondroitin sulfate are sold as dietary or nutritional supplements. They can be extracted from animal tissue: glucosamine from crab, lobster or shrimp shells; and chondroitin sulfate from animal cartilage, such as tracheas or shark cartilage.

Ginger is one of the oldest known natural anti-inflammatories. There is data on ginger decreasing inflammation in arthritic knees. Ginger has also shown to have effect on decreasing cardiac vessel inflammation. Ginger is a cyclooxygenase inhibitor. Ginger has a relative rapid onset of action for controlling pain. Adding ginger to glucosamine has shown to have enhanced clinical effect in a clinical practice with over six hundred arthritic knees and hips with rapid onset of clinical effect of pain control. The only drawback to ginger is that it can cause heartburn in approximately 5% of patients who cannot tolerate taking it. Otherwise both ginger and glucosamine have been used safely with very low side-effect profile.

Ginger, the underground stem, or rhizome, of the plant Zingiber officinale has been used as a medicine in Asian, Indian, and Arabic herbal traditions since ancient times. Ginger products are made from fresh or dried ginger root, or from steam distillation of the oil in the root. The herb is available in extracts, tinctures, capsules, and oils. The important active components of the ginger root are thought to be volatile oils and pungent phenol compounds (such as gingerols and shogaols). Typically, the ginger used in the current invention is dried ginger root or ginger oil.

Ginger functions as a circulatory stimulant to relax peripheral blood vessels thus serving the dual beneficial roles of removing detrimental inflammatory byproducts such as free radicals and transporting an ample supply of anti-oxidants and metabolic precursor building blocks for repair.

Ginger also functions as an antioxidant. Oxygen-derived free radicals apparently act as mediators of inflammation and/or tissue destruction in inflammatory and arthritic disorders. Free radicals degrade hyaluronic acid, modify collagen and perhaps proteoglycan structure and/or synthesis, alter and interact with immunoglobulins, activate degradative enzymes and inactivate their inhibitors, and possibly participate in chemotaxis. It is reasonable to conclude that a composition containing antioxidants would be advantageous in that the free radicals could be scavenged and detoxified before they reached the affected area.

Methylsulfonylmethane, abbreviated MSM, is an organic sulfur-containing compound that occurs naturally in a variety of fruits, vegetables, grains and in animals, including humans in at least trace amounts. MSM has also been found in such plants as Equisetem arvense, also known as horsetail. The biological role of MSM, if any, is not known. MSM is a metabolite of dimethyl solfoxide or DMSO (see Dimethyl Sulfoxide). It is believed that some of the possible effects of DMSO could be attributed to MSM.

### EXAMPLE

In our clinical study data in six hundred and thirty three arthritic knees and hips, a daily dose of 510 mg ginger, 1500 mg glucosamine and 1200 mg chondroitin sulfate (Group 1) has shown to have better pain control (p<0.05) with quicker onset of pain control (p<0.05) when compared to a daily dose of a combination of 1500 mg glucosamine and 1200 mg chondroitin sulfate (Group 2). Both groups were age, sex and weight matched. Group 1 had 350 patients while Group 2 had 293 patients. The patients were chosen to be in Group 1 or Group 2 based on their choice. Seventeen patients dropped out of Group 1 and 7 patients dropped out of Group 2 due to their inability to tolerate the supplements. A pain scale was used to collect pain data with 0 being no pain and 10 being worst pain. Patients were evaluated at 1 week, and again at 3 months, after starting the supplements. Group 1 reported pain score of 7.3 pre-treatment and 4.1 at one week and 3.4 at 3 months. Group 2 reported pain score of 7.2 pre-treatment and 5.5 at one week and 4.3 at 3 months. The study clearly shows that adding 510 mg ginger to the traditional combination of 1500 mg glucosamine and 1200 mg chondroitin sulfate has superior pain control with earlier onset of pain control.

Similarly, clinical trial studies comparing a daily dose of 1500 mg glucosamine combined with 1200 mg methylsulfonylmethane (MSM) in 37 patients (Group 3) compared with an age and sex matched group of patients taking a daily dose of 1500 mg glucosamine, 1200 mg MSM and 510 mg ginger in 74 patients (Group 4) has shown us the following results: Group 3 reported pain score of 7.3 pre-treatment and 4.9 at one week and 4.2 at 3 months. Group 1 reported pain score of 7.4 pre-treatment and 3.9 at one week and 3.2 at 3 months. Group 4 using ginger similarly reported 5% incidence of mild stomach irritation.

Our invention is based on this clinical observation. It is using ginger in the range of 510-2000 mg (510 mg preferably) along with glucosamine in the range of 1000-2500 mg daily (1500 mg daily preferably) to create an enhanced pain control with quicker onset of pain control. The supplement can be administered in capsule form with each capsule containing 375 mg of glucosamine, 300 mg of chondroitin sulfate, and 127.5 mg of ginger. The daily recommended dose for the capsules is two capsules twice a day to give daily totals of 1500 mg of glucosamine, 510 mg of ginger and 1200 mg of chondroitin sulfate.

What is claimed is:

1. A pain-controlling composition comprising at least about 510 mg of ginger, at least about 1000 mg of glucosamine and at least about 1200 mg of chondroitin sulfate or methylsulfonylmethane, all weights based on a total daily dosage of the composition.

2. The composition of claim 1 wherein the ginger is present in an amount between about 510 mg and about 2000 mg, based on the total daily dosage of the composition.

3. The composition of claim 1 wherein the ginger is present in an amount of about 510 mg based on the total daily dosage of the composition.

4. The composition of claim 1 wherein the glucosamine is present in an amount between about 1000 mg to about 2500 mg, based on the total daily dosage of the composition.

5. The composition of claim 1 wherein the glucosamine is present in an amount of about 1500 mg based on the total daily dosage of the composition.

6. The composition of claim 1 wherein the ginger is present in an amount of about 510 mg and the glucosamine is present in an amount of about 1500 mg, all weights based on the total daily dosage of the composition.

7. A therapeutics delivery system comprising at least one capsule containing a composition comprising at least about 510 mg of ginger, at least about 1000 mg of glucosamine and at least about 1200 mg of chondroitin sulfate or methylsulfonylmethane, all weights based on a total daily dosage of the composition.

8. The delivery system of claim 7 wherein the daily dosage is divided among at least 4 capsules.

9. The delivery system of claim 7 wherein the ginger is present in an amount between about 510 mg and about 2000 mg, based on the total daily dosage of the composition.

10. The delivery system of claim 7 wherein wherein the glucosamine is present in an amount between about 1000 mg to about 2500 mg, based on the total daily dosage of the composition.

11. The delivery system of claim 7 wherein the ginger is present in an amount of about 510 mg and the glucosamine is present in an amount of about 1500 mg, all weights based on the total daily dosage of the composition.

12. A method for controlling pain, the method comprising ingesting a daily dosage of a composition that comprises at least about 510 mg of ginger, at least about 1000 mg of glucosamine and at least about 1200 mg of chondroitin sulfate or methylsulfonylmethane, all weights based on a total daily dosage of the composition.

13. The method of claim 12 wherein the ginger is present in an amount between about 510 mg and about 2000 mg, based on the total daily dosage of the composition.

14. The method of claim 12 wherein wherein the glucosamine is present in an amount between about 1000 mg to about 2500 mg, based on the total daily dosage of the composition.

15. The method of claim 12 wherein the ginger is present in an amount of about 510 mg and the glucosamine is present in an amount of about 1500 mg, all weights based on the total daily dosage of the composition.

16. The method of claim 12 wherein the daily dosage is divided into at least two partial dosages and the partial dosages are ingested at separate times during the day.

17. The method of claim 12 wherein the daily dosage of the composition is contained within at least one capsule.
